(19) United States
(12) Plant Patent Application Publication
(10) Pub. No.: US 2005/0183179 P1
(43) Pub. Date: Aug. 18, 2005

(54) Chrysanthemum plant 'Sunny Spider'

(50) CHRYSANTHEMUM MORIFOLIUM; CHRYSANTHEMUM

(76) Inventor: Keith G. Lintott, Bognor Regis (GB)

Correspondence Address: Mark K. Dickson, Suites 200 & 300, 2941 Fairview Park Drive, Falls Church, VA, 22042, US

(21) Appl. No.: 11/055,171

(22) Filed: Feb. 9, 2005

Related U.S. Application Data
(60) Provisional application No. 60/544,382, filed on Feb. 12, 2004.

(51) Int. Cl. A01H 5/00

(52) U.S. Cl. PLT/289

(57) ABSTRACT

A new variety of Chrysanthemum plant named ‘Sunny Spider,’ having a domed capitulum of bright yellow spider decorative flowers and a round shape and a uniform habit. The flowers last well under adverse weather conditions.

This application claims the benefit of U.S. Provisional Application No. 60/544,382, filed on Feb. 12, 2004.

### LATIN NAME OF THE GENUS AND SPECIES

Botanical classification: Chrysanthemum morifolium.

### VARIETY DENOMINATION

The new Chrysanthemum variety denomination is ‘Sunny Spider.’

### BACKGROUND OF THE INVENTION

The present invention comprises a new and distinct cultivar of Chrysanthemum botanically known as Chrysanthemum morifolium, and referred to by the variety name ‘Sunny Spider.’

‘Sunny Spider,’ identified as 30859-0, originated from a cross made in a controlled breeding program in Chichester, West Sussex, United Kingdom.

The female parent of the new Chrysanthemum variety is the variety known as ‘Sarah’ U.S. Plant Pat. No. 7586. The male parent of the new Chrysanthemum variety is an undistributed, unpatented seedling identified as ‘G26C23.’ The new Chrysanthemum variety ‘Sunny Spider’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Sunny Spider’ is a garden type of Chrysanthemum plant variety having a domed of golden yellow spider decorative type of flowers.

### Comparison with Female Parent

Plants of the new Chrysanthemum variety ‘Sunny Spider’ are dissimilar to the parent variety ‘Sarah’ in plant habit and growth rate. In side-by-side comparisons in Chichester, West Sussex, United Kingdom, under commercial practice, plants of the new Chrysanthemum variety ‘Sunny Spider’ differed from plants of the parent variety ‘Sarah’ in the following characteristics.

1. The new Chrysanthemum variety ‘Sunny Spider’ produces golden yellow spider decorative flowers whereas the parent variety ‘Sarah’ produces bronze spider decorative flowers.

2. Plants of the new Chrysanthemum variety ‘Sunny Spider’ have larger and shorter inflorescence than plants of the parent variety ‘Sarah.’

3. Plants of the new Chrysanthemum variety ‘Sunny Spider’ are less upright in growth habit than plants of parent variety ‘Sarah.’

### Comparison with Male Parent

Plants of the new Chrysanthemum variety ‘Sunny Spider’ are dissimilar to the parent variety ‘G26C23’ in plant habit and growth rate. In side-by-side comparisons in Chichester, West Sussex, United Kingdom, under commercial practice, plants of the new Chrysanthemum variety ‘Sunny Spider’ differed from plants of the parent variety ‘G26C23’ in the following characteristics.

1. The new Chrysanthemum variety ‘Sunny Spider’ produces yellow spider decorative flowers whereas the parent variety ‘G26C23’ produces pink quilled petal type decorative flowers.

2. Plants of the new Chrysanthemum variety ‘Sunny Spider’ have a more rounded and domed habit than plants of the parent variety ‘G26C23.’

### Comparison with Other Variety

Plants of the new Chrysanthemum variety ‘Sunny Spider’ are dissimilar to the variety ‘Yellow Sarah,’ U.S. Plant Pat. No. 8804, in plant habit and growth rate. In side-by-side comparisons in Chichester, West Sussex, United Kingdom, under commercial practice, plants of the new Chrysanthemum variety ‘Sunny Spider’ differed from plants of the variety ‘Yellow Sarah’ in the following characteristics.

1. The new Chrysanthemum variety ‘Sunny Spider’ produces golden yellow spider decorative flowers whereas the variety ‘Yellow Sarah’ produces yellow quilled petal type decorative flowers.

2. Plants of the new Chrysanthemum variety ‘Sunny Spider’ have a more rounded and domed habit than plants of the variety ‘Yellow Sarah.’

### BRIEF DESCRIPTION OF ILLUSTRATION

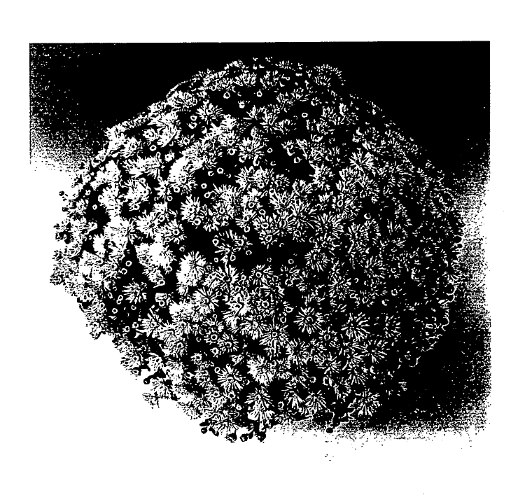

Typical specimens of the plant and flowers for the new Chrysanthemum variety ‘Sunny Spider’ are shown in the accompanying digital photograph. The colors shown are as true as possible within the usual limits of this kind of illustration. FIG. 1 is a whole plant view of the new Chrysanthemum variety ‘Sunny Spider’ grown in a pot. The plant shown in the illustration is 65 days from the commencement of planting outdoors in mid-June.

### DETAILED BOTANICAL DESCRIPTION

The following description of the new Chrysanthemum variety ‘Sunny Spider’ is of plants grown outside, natural season, in Chichester, West Sussex, United Kingdom in the month of June. The variety has not been observed under all possible environmental conditions. The phenotype may vary significantly with variations in the environment such as temperature, length of day and light intensity, without any variance in genotype. The commercial classification of the new variety is a garden Chrysanthemum.

Plants of the new Chrysanthemum variety have been grown successfully under temperature conditions averaging about 50° F. at night and about 60° F. to 75° F. during the day under natural light conditions. The plants were grown outdoors in mid-June in 4 liter pots using loamless compost. No growth retardants were used. To produce a commercial product the plants may be pinched once with the center bud removed. The typical container size for commercial growth is 4 liters (8″). It has been observed that the shelf life of the new variety is about 26 days with a response time of about 9 weeks natural season. The new variety is suitable for growth in a temperature range of 50° F. to 75° F.

The new variety may be produced as a pot plant. The following description is with respect to a plant produced as garden pot plant. In the description of this new Chrysanthemum variety, color values have been taken from The Royal Horticultural Society Colour Chart (R.H.S.C.C.).

### Plant

Plant type: Pot.
Habit: Domed-Round.
Height: 50 cm-55 cm.
Width: 50 cm-55 cm.
Branching characteristics: Free branching and spreading.
Length of lateral branches: 10 cm-16 cm.
Number of breaks from pinch: 7-11.
Stem
   Length.—7 cm-9 cm.
   Diameter.—0.5 cm-0.7 cm.
   Texture.—Rough.
   Color.—137A.
Response time: near 9 weeks natural season.
Vigor: Medium.
Shelf life: near 26 days.
Disease (susceptibility/resistance observed): None observed to date.
Pest (susceptibility/resistance observed): None observed to date.
Drought and temperature (susceptibility/resistance): None observed to date.
Growth retardant type and treatment: No treatment necessary.
The plants were grown for 9 weeks in natural season conditions. Propagation:
   Type.—Vegetative Propagation via Stem Cuttings.
   Time to rooting.—12 days with soil temperatures of 70° F.
   Rooting habit.—Fine and Fibrous.

### Foliage

Number of leaves per lateral branch: 7-12.
Compound or single: Single.
Arrangement of leaves: Alternate.
   Shape of leaf.—Typically 5 lobed ovate.
   Size of leaf.—Width: 3 cm-4 cm Length: 4 cm-6 cm.
   Leaf apex: Acute.
   Base: Obtuse.
   Attachment: Petioled.
   Aspect: Slightly undulating.
   Margin: Palmately lobed.
   Surface characteristics.—Top: Slightly Pubescent Bottom: Slightly Pubescent.
Petiole: Color: near 147B Length: 0.6 cm-1 cm.
Venation: Net. Prominent mid vein at underside Color: upper side: near 138C under side: near 138C.
Color: Mature Leaf, upper side: near 147A; under side: near 147B Young Leaf, upper side: near 147A; under side: near 147B

### Flower

Flower appearance: Matte.
Flower type: Spider Decorative.
Flower form: Flat.
Flower shape: Round.
Flowering habit: Cyme.
Number of blossoms per branch: 5-9.
Inflorescence form: Corymbiform.
   Depth of fully expanded blossoms.—1.5 cm-2 cm.
   Diameter of fully expanded blossoms.—4.0 cm-6.0 cm.
Phyllaries:
   Number.—22-27.
   Color.—near 137A.
   Length.—0.4 cm-0.6 cm.
   Width.—0.15 cm-0.3 cm.
   Texture/appearance.—Slightly Pubescent/Dull.
Peduncle: Peduncle length is shortest at the terminal flower and progressively longer down the stem; longest length is on average 9 cm-11 cm, shortest length is on average 5 cm-6 cm.
   Length.—5 cm-11 cm.
   Color.—near 138A.
   Surface.—Slightly Pubescent.
   Habit.—Slightly curving.
   Strength.—Strong.
Ray florets:
   Form/shape.—Quilled.
   Texture/appearance.—Matte.
   Number per flower.—200-225.
   Length.—1 cm-3 cm; Width: 0.2 cm-0.4 cm.
   Apex.—Rounded.
   Base.—Tubular.
   Margin.—Entire.
Disc florets: none observed.
Fragrance: Slight.
Flower bud (at onset of color):
   Length.—0.4 cm-0.7 cm.
   Diameter.—0.5 cm-0.8 cm.
   Form/shape.—Ball shaped slightly flattened.
General flower color:
   1. Ray florets, upper side.—Immature: near 3B Mature: near 3D Older/Fading: near 2D.
   2. Ray florets, under side.—Immature: near 3B Mature: near 3D Older/Fading: near 2D.
   3. Bud.—near 139C.
Flower progression with age; Flowers hold the yellow color well with age.
The lastingness of blooms is about 26 days.

### Reproductive Organs

Gynoecium present on Ray florets only. Ray florets per individual flower:
   Pistil number.—80-100 observed.
   Stigma color.—near 10A.
   Stigma shape.—Forked.
   Style color.—near 1C.
   Style length.—0.2 cm.
Androecium not observed.
Fruit and seeds: None observed.

1. A new and distinct variety of Chrysanthemum plant, substantially as described and illustrated herein.
